(19) United States
(12) Patent Application Publication
(10) Pub. No.: US 2026/0294686 A1
(43) Pub. Date: Oct. 1, 2026

(54) SYSTEM AND METHOD FOR GUIDING LASER-ASSISTED SURGERY BASED ON FLEXIBLE RANGE OPTICAL COHERENCE TOMOGRAPHY

(71) Applicant: Alcon Inc., Fribourg (CH)

(72) Inventors: Wei Wei, Lake Forest, CA (US); Gangjun Liu, Portland, OR (US); Lingfeng Yu, Rancho Santa Margarita, CA (US)

(21) Appl. No.: 19/568,989

(22) Filed: Mar. 17, 2026

Related U.S. Application Data
(60) Provisional application No. 63/781,414, filed on Apr. 1, 2025.

(51) Int. Cl. A61F 9/008

(52) U.S. Cl. CPC A61F 9/008; A61F 2009/00851

(57) ABSTRACT

A system for guiding an ophthalmic procedure includes an optical coherence tomography (OCT) device configured to obtain respective data of a target site in an eye. The OCT device has a swept source laser tunable through a range of wavelengths. The OCT device includes a first imaging mode with a first imaging depth and a second imaging mode with a second imaging depth, the first imaging depth being greater than the second imaging depth. The controller is adapted to execute first and second sampling modules to process the respective OCT signal from the first and second imaging modes. The controller is adapted to generate an output signal indicative of at least one of pupil centering information and tilt angle of the target site based in part on the first dataset and the second dataset. The output signal is employed for pre-surgical alignment of the target site.

### INTRODUCTION

The disclosure relates generally to a guidance system for laser-assisted ophthalmic surgery based on flexible range Optical Coherence Tomography (“OCT”). Ophthalmic surgery encompasses a range of surgical procedures for treating conditions affecting the eye, from common refractive errors to complex retinal disorders. Modern advances in surgical techniques, such as laser technology, offers enhanced precision in creating surgical incisions and delivering more consistent and reproducible outcomes. However, precisely aligning and docking the laser apparatus prior to and during surgery remains a non-trivial challenge.

### SUMMARY

A system for guiding an ophthalmic procedure includes an optical coherence tomography (OCT) device configured to obtain respective data of a target site in an eye, including a respective OCT signal. The OCT device has a swept source laser tunable through a range of wavelengths. A controller is in communication with the OCT device. The controller has a processor and tangible, non-transitory memory on which instructions are recorded. The OCT device includes a first imaging mode with a first imaging depth and a second imaging mode with a second imaging depth, the first imaging depth being greater than the second imaging depth.

The controller is adapted to execute a first sampling module to process the respective OCT signal from the first imaging mode into a first dataset at a first sampling frequency. A second sampling module is executed to process the respective OCT signal from the second imaging mode into a second dataset at a second sampling frequency. The controller is adapted to generate an output signal indicative of at least one of pupil centering information and tilt angle of the target site based in part on the first dataset and the second dataset. The output signal is employed for pre-surgical alignment of the target site.

In some embodiments, the first imaging depth is between about 30 and 60 millimeters, and the second imaging depth is between about 8 and 30 millimeters. The ophthalmic procedure employs a laser delivery system in communication with the controller. In some embodiments, a docking device is adapted to interface between the eye and the laser delivery system. The first imaging mode is employed when the docking device is in a pre-docking position at a predefined distance from the eye and the second imaging mode is employed when the docking device is in a docking position adjacent to the eye. The controller is adapted to align the docking device relative to the target site based in part on the output signal.

The first imaging mode and the second imaging mode are executed with a first pulse repetition rate and a second pulse repetition rate, respectively, of the swept source laser. The controller may be adapted to set the first pulse repetition rate to be about half of the second pulse repetition rate when the first sampling frequency and the second sampling frequency are close in value. The controller may be adapted to set the first pulse repetition rate to be relatively close in value to the second pulse repetition rate when the first sampling frequency is at least twice of the second sampling frequency. In some embodiments, the first pulse repetition rate is about 50 kilo Hertz, and the second pulse repetition rate is about 100 kilo Hertz, the second imaging mode having an equal or higher axial resolution relative to the first imaging mode.

In some embodiments, a femtosecond laser device operatively connected to the docking device and adapted to generate a treatment beam, the controller being adapted to guide the treatment beam towards the target site based in part on the output signal. An interferometer is embedded in the OCT device and adapted to transmit a k-clock signal to the controller. In some embodiments, a first analog-to-digital converter and a second analog-to-digital converter are operatively connected to the OCT device. The first analog-to-digital converter and the second analog-to-digital converter operate in an interleaved fashion with a predefined delay relative to one another. The first analog-to-digital converter is adapted to sample the respective OCT signal based in part on respective rising edges of the k-clock signal, and the second analog-to-digital converter is adapted to sample the respective OCT signal based on respective falling edges of the k-clock signal.

In some embodiments, an interferometer embedded in the OCT device and adapted to transmit a first k-clock signal and a second k-clock signal to the controller. Here, the first sampling module is adapted to sample the respective OCT signal based on the first k-clock signal, and the second sampling module is adapted to sample the respective OCT signal based on the second k-clock signal. The first k-clock signal is defined by a first frequency and the second k-clock signal is defined by a second frequency different from the first frequency. A digitizer is operatively connected to the OCT device, the digitizer having an internal clock. In some embodiments, the controller is adapted to acquire an OCT signal from the OCT device using the internal clock of the digitizer, and resample the OCT signal to equally k-spaced samples.

Disclosed herein is a method for guiding an ophthalmic procedure in a system having an optical coherence tomography (OCT) device and a controller with at least one processor and at least one non-transitory, tangible memory. The method includes receiving respective data of a target site in an eye via a first imaging mode of the OCT device, the first imaging mode having a first imaging depth, via the controller, the respective data including a respective OCT signal. The OCT device has a swept source laser tunable through a range of wavelengths. The method includes receiving the respective data of the target site via a second imaging mode of the OCT device, via the controller. The second imaging mode has a second imaging depth, the first imaging depth being greater than the second imaging depth.

The method includes executing a first sampling module to process the respective OCT signal from the first imaging mode into a first dataset at a first sampling frequency. The method includes executing a second sampling module to process the respective OCT signal from the second imaging mode into a second dataset at a second sampling frequency. The method includes generating an output signal indicative of at least one of a pupil centering information and tilt angle of the target site based on the first dataset and the second dataset, via the controller. The method includes aligning a docking device with the target site prior to surgery based on the output signal.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 is a schematic example illustration of a system for guiding laser-assisted ophthalmic surgery, the system having a controller;

FIG. 2 is a schematic flowchart for a method executable by the controller of FIG. 1;

FIG. 3 is a schematic example illustration of a docking device for a laser delivery system in a pre-docking position and a docking position; and

FIGS. 4-7 are schematic example graphs of various k-clock waveforms and time-sequential samples acquired by the controller of FIG. 1.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### DETAILED DESCRIPTION

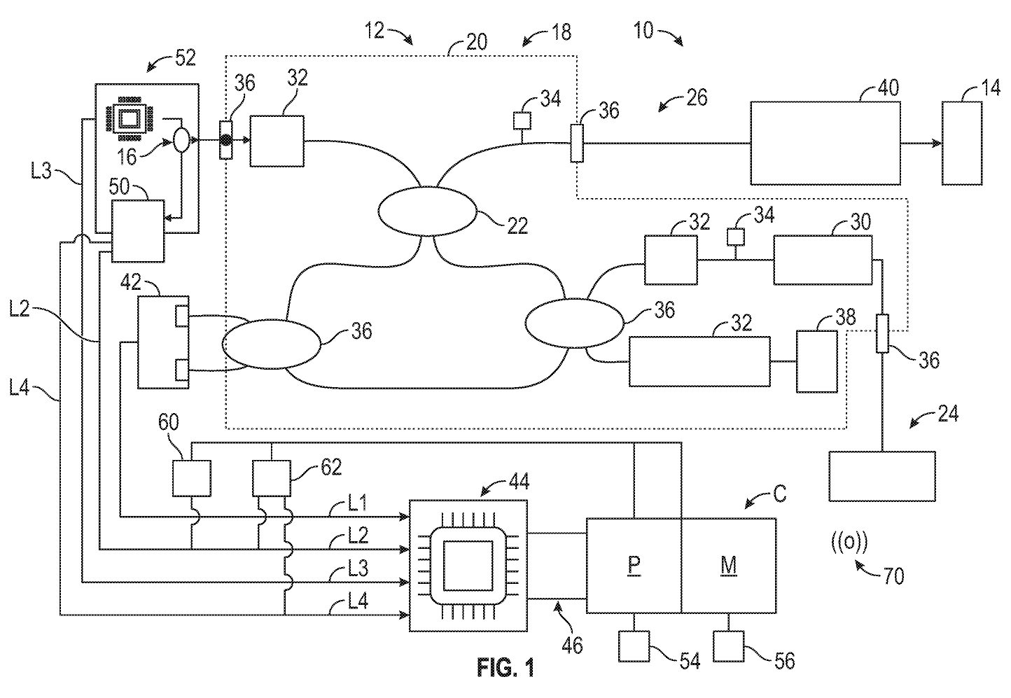

Referring to the drawings, wherein like reference numbers refer to like components, FIG. 1 schematically illustrates a system 10 for guiding laser-assisted ophthalmic surgery, both prior to and during surgery. Referring to FIG. 1, the system 10 includes an optical coherence tomography (OCT) device 12 configured to obtain respective data of a target site 14 in an eye. The OCT device 12 includes a swept source laser 16 tunable through a range of wavelengths.

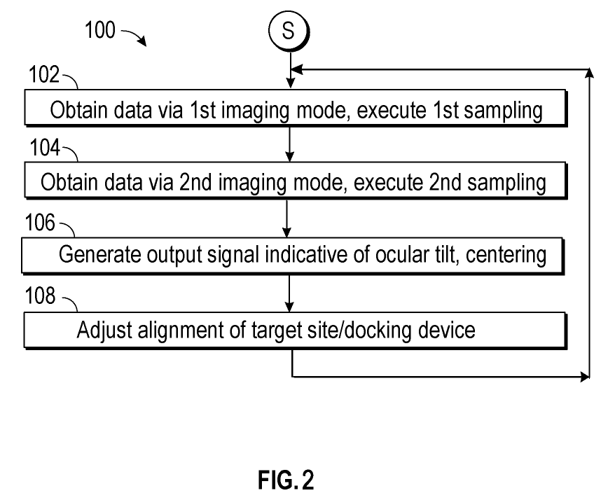

Referring to FIG. 1, the system 10 includes a controller C having at least one processor P and at least one memory M (or non-transitory, tangible computer readable storage medium) on which instructions are recorded for executing a method 100 for guiding assistance in a laser-assisted ophthalmic surgery, both prior to and during surgery. Method 100 is shown in and described below with reference to FIG. 2.

Modern advances in surgical techniques, such as laser technology, offers enhanced precision in creating surgical incisions and delivering more consistent and reproducible outcomes. For example, in laser-assisted cataract surgery, lasers are employed to perform precise incisions and lens fragmentation. However, precisely aligning and docking the laser apparatus prior to and during cataract surgery remains a non-trivial challenge. Here there are two mechanical tasks that need to be accomplished in docking: immobilization of the eyeball and connection of the eye to the femtosecond laser system. A docking device is employed to simultaneously achieve these two independent mechanical tasks, making docking further challenging.

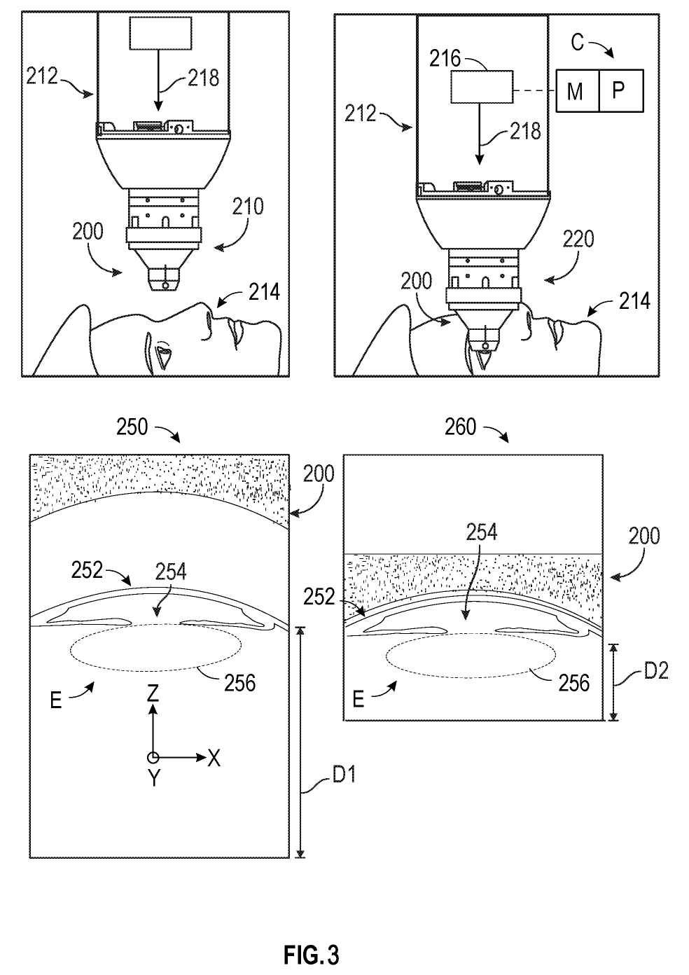

An example docking device 200 for a laser delivery system 212 is shown in FIG. 3. FIG. 3 illustrates a pre-docking position 210 at a predefined distance from the eye E and a docking position 220 adjacent to the eye E. The docking device 200 serves as a contacting element that establishes the connection between the laser delivery system 212 and the eye E of the patient 214, creating a base or foundation for laser energy delivery.

As described below, the OCT device 12 offers at least two OCT imaging modes of different imaging depths. The depth-extended OCT device 12 may be employed during pre-docking and docking procedures in laser-assisted surgery, such as laser-assisted cataract surgery. This enhanced capability allows for continuous monitoring of targets of interest while simultaneously providing precise depth measurements for positioning.

In the embodiment shown in FIG. 3, a first imaging mode 250 of the OCT device 12 is employed when the docking device 200 is in a pre-docking position 210 a predefined distance (e.g., 10-30 centimeters) from the eye E. Referring to FIG. 3, a second imaging mode 260 of the OCT device 12 is employed when the docking device 200 is in a docking position 220 adjacent to the eye E (e.g., at cornea 252) of the patient 214. The first imaging mode 250 has a first imaging depth D1 and the second imaging mode 260 has a second imaging depth D2. The first imaging depth D1 is greater than the second imaging depth D2. As described below, the first imaging mode 250 operates in part by increasing the number of sampling points across the wavelength sweep of the swept source laser 16.

As noted above, the first and second imaging modes 250, 260 shown in FIG. 3 collect respective data that is subsequently processed by the controller C. Referring to FIG. 1, the controller C is adapted to execute a first sampling module 54 and a second sampling module 56 to process the respective data from the first and second imaging modes 250, 260 into a first dataset and a second dataset, respectively. As will be described below, the second sampling module 56 is configured to capture data points at twice a frequency relative to the first sampling module 54. The controller C is adapted to generate an output signal based on the first dataset and the second dataset. The output signal may indicate centering information of the pupil 254 and/or lens 256 of the eye E relative to a predefined XYZ axis, shown in FIG. 3. The target site is the lens 256 in cataract surgery. The output signal may indicate the tilt angle of the lens 256. The tilt angle may be along a depth axis (Z axis), lateral axis (Y axis) or a longitudinal axis (X axis).

The output signal is used to adjust the positioning of the docking device 200 in FIG. 3. Referring to FIG. 3, the controller C is adapted to generate an alignment signal to align the docking device 200 relative to the eye E based on the output signal. For example, the docking device 200 may be moved to adjust to the tilt or skew of the lens 256. Additionally, when the docking device 200 shifts or the eye E moves slightly, the controller C provides real-time feedback, facilitating prompt corrective actions.

In some embodiments, the first imaging depth D1 is between about 30 and 60 millimeters, and the second imaging depth D2 is between about 8 and 30 millimeters. In other embodiments, the first imaging depth D1 is between about 60 and 120 millimeters, and the second imaging depth D2 is between about 8 and 30 millimeters. The second imaging mode 260 has an equal or higher axial resolution relative to the first imaging mode 250.

FIG. 3 illustrated a first imaging mode 250 and a second imaging mode 260. The first imaging mode 250 is executed with a first pulse repetition rate of the swept source laser 16, while the second imaging mode 260 is executed with a second pulse repetition rate of the swept source laser 16. The switch between the two imaging modes may be made within about 100 milliseconds or faster.

Referring to FIG. 3, the laser delivery system 212 may include a femtosecond laser device 216 adapted to generate a treatment beam 218 for the patient 214. The treatment beam 218 may include a plurality of ultra-short laser pulses, each having a time duration of between about a femtosecond (10⁻¹⁵ seconds) and about 50 picoseconds (50 x 10⁻¹² seconds). In one example, the femtosecond laser device 216 is configured to deliver infrared radiation at a wavelength of between about 700 nanometers and 1220 nanometers.

Referring now to FIG. 1, the OCT device 12 includes a swept source laser 16 tunable through a range of wavelengths. In one example, the swept source laser may be a 1060 nm VCSEL Swept-Wavelength Laser Sources. A single sweep is referred to as an A-scan, with multiple A-scans are performed at various locations to produce slice (2D) or volume (3D) scans, referred to as B-scan and C-scan respectively. The swept-source laser wavelength may be increased or decreased within each A-scan in a number of steps. The step size is non-linear and may increase or decrease with wavelength. The laser output may be directed to multiple channels with variance delay in order to cover different scanning ranges.

Referring to FIG. 1, the laser output travels to an integrated interferometer 18 sealed in a mechanical housing 20. Within the housing 20, a beam coupler 22 may be employed to divide the light between a reference arm 24 and a sample arm 26. The reference arm 24 includes a reference delay line 30 that adjusts a reference arm length to match an optical path delay of the sample arm 26. The OCT device 12 may include one or more variable optical attenuators 32 to control the power of the optical signal, thereby preventing detector saturation. The housing 20 may further include respective polarization controllers 34 adapted to adjust light polarization in both arms, optical couplers 36, and a photodetector 38.

Referring to FIG. 1, the sample arm 26 is connected to an imaging head 40, which includes mechanisms to adjust OCT beam zoom capability, focus, and an objective lens system (not shown). The interference pattern between the return signals from the sample arm 26 and reference arm 24 is detected by a balanced photo detector 42 which converts incoming optical signals into electrical signals. A balanced photodetector 42 is employed to reduce common-mode noise and enhances signal quality by using two detectors to measure differential signals.

Referring to FIG. 1, the balanced photo detector 42 transmits the OCT signal (indicated by L1) to a digitizer 44, which transmits the data to the controller C via an interconnecting device 46. The controller C may execute a postprocessing module on the raw OCT data to generate the cross-sectional OCT images. This process may include background removal, spectral windowing, dispersion compensation, fast Fourier transform, and logarithmic compression.

Referring to FIG. 1, an interferometer 50, such as a built-in Mach Zehnder interferometer 50, is embedded within the OCT device 12 and adapted to generate an external k-clock signal (indicated by line L2 in FIG. 1) transmitted to the controller C. The interferometer 50 may be housed within a swept laser module 52 alongside the swept source laser 16. The swept laser module 52 includes various circuitry and electronic components available to those skilled in the art and transmits an A-trigger signal (indicated by line L3 in FIG. 1) to the controller C. The system 10 utilizes a variable frequency sampling clock, referred to herein as a k-clock, which synchronizes directly with the changing wavelength of the swept source laser 16. This “k” designation indicates that the clock signal maintains linearity in wave number (k) space (2π /wavelength) while exhibiting non-linearity in the time domain.

Referring now to FIG. 2, a flow chart of the method 100 is shown, which may be embodied as computer-readable code or instructions stored on and executable by the controller C of FIG. 1. Method 100 need not be applied in the specific order recited herein and some blocks may be omitted. The memory M can store controller-executable instruction sets, and the processor P can execute the controller-executable instruction sets stored in the memory M.

Per block 102 of FIG. 2, the method 100 includes obtaining the respective OCT signal from the OCT device 12 in the first imaging mode 250 and executing a first sampling module 54 to process the respective OCT signal into a first dataset at a first sampling frequency. Per block 104 of FIG. 2, the method 100 includes obtaining the respective OCT signal from the OCT device 12 in the second imaging mode 260 and executing a second sampling module 56 to process the respective OCT signal into a second dataset at a second sampling frequency. In some embodiments, the first sampling module 54 may operate at both rates to sample the dataset(s). As noted above, the first imaging mode 250 and the second imaging mode 260 are executed with a first pulse repetition rate and a second pulse repetition rate, respectively, of the swept source laser 16. The controller C is adapted to set the first pulse repetition rate to be the same as or relatively close in value to the second pulse repetition rate (e.g., within about 1% of each other) when the first sampling frequency is at least twice of the second sampling frequency. The controller C is adapted to set the first pulse repetition rate to be about half of the second pulse repetition rate when the first sampling frequency and the second sampling frequency are about the same as or relatively close in value e.g., within about 1% of each other). For example, the first pulse repetition rate may be about 50 kilo Hertz, and the second pulse repetition rate may be about 100 kilo Hertz when the first sampling frequency and the second sampling frequency are the same.

The imaging depth of the OCT device 12 may be adjusted according to the parameters of a defined k-space. K-space or wavenumber space refers to a mathematical space where each point represents a specific spatial frequency of a wave, describing how many wave cycles occur per unit distance within a system, with the “k” value representing the wavenumber which is calculated as 2π divided by the wavelength. In other words, each specific wavelength is identified by its wavenumber, denoted k, and the k-clock output has a frequency corresponding to the swept wavelength of the laser source. Due to its non-linear nature, the k-clock frequency may vary during the A-scan.

Three embodiments are described below for processing the data. The choice between these sampling approaches depends on several factors, including the required imaging speed, depth range, and system complexity constraints. The k-clock signal is used to guide where (e.g. according to the position of the rising edges or falling edges) the sampling modules are to sample the OCT signal. In other words, the sampling modules will sample the OCT signal that coincides with the rising and/or falling edge positions of the k-clock signal.

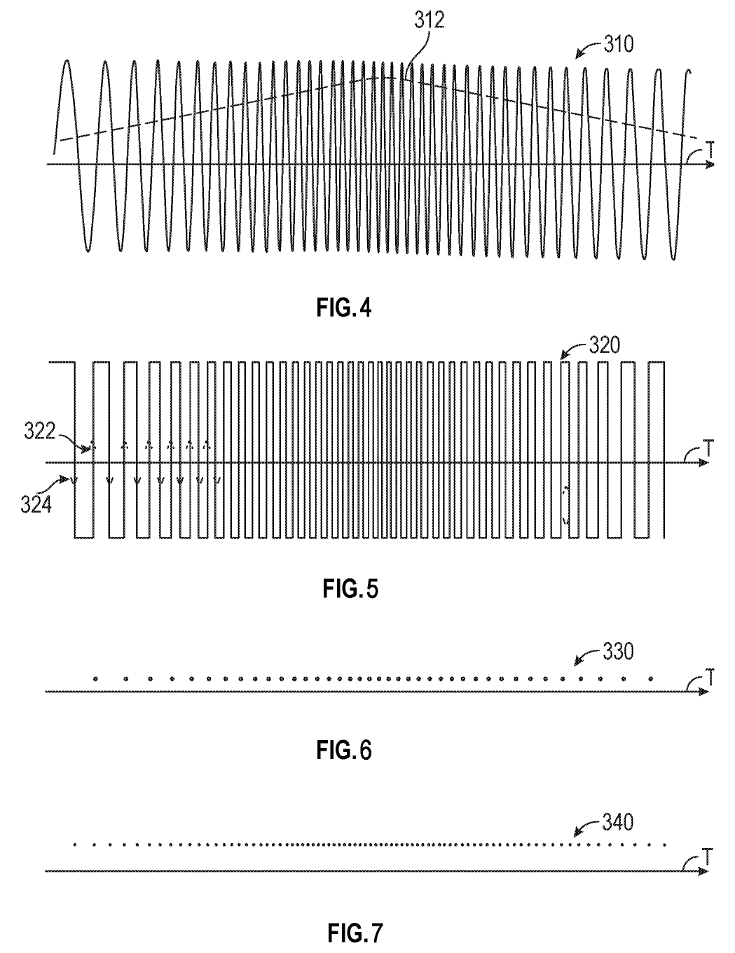

An analog k-clock signal (with a continuously varying signal) and a digital k-clock signal (with discrete steps) are shown in FIGS. 4-5. FIG. 4 illustrates an example waveform 310 of an analog k-clock over time (T), the waveform 310 being defined by frequency trace 312. FIG. 5 illustrates an example waveform 320 of a digital k-clock over time (T), with rising edges 322 and falling edges 324. Referring to FIG. 5, in a first embodiment, a dual edge clocking approach is employed. Here the controller C is adapted to clock the dual channels of the digitizer 44 using the rising edges 322 and falling edges 324, respectively. The dual-edge k-clocking extends imaging depth by up to a factor of two compared to single-edge clocking.

Referring to FIG. 1, the system 10 may include a first analog-to-digital converter 60 and a second analog-to-digital converter 62 operatively connected to the OCT device 12. The first analog-to-digital converter 60 is adapted to sample the respective OCT signal coinciding with or based on respective rising edge positions of the k-clock signal (indicated by line L2 in FIG. 1), and the second analog-to-digital converter 62 is adapted to sample the respective OCT signal coinciding with or based on respective falling edge positions of the k-clock signal. The first and the second analog-to-digital converters 60, 62 operate in an interleaved fashion with a predefined delay relative to one another. This technique acquires two samples per clock cycle.

The contrast between single edge k-clocking and dual edge k-clocking is shown in FIGS. 6-7. FIG. 6 illustrates an example graph of time-sequential samples 330 acquired with single edge k-clocking through the digitizer 44 over time (T). FIG. 7 illustrates an example graph of time-sequential samples 340 acquired with dual edge k-clocking through the digitizer 44 over time (T).

Referring now to a second embodiment, a swept source laser 16 with two different k-clocking frequencies may be employed. Here, the interferometer 50 is adapted to transmit a first k-clock signal (indicated by line L2 in FIG. 1) defined by a first frequency, and a second k-clock signal (indicated by line L4 in FIG. 1) to the controller C. The second k-clock signal is defined by a second frequency that is different from the first frequency. The first sampling module 54 is adapted to sample the respective OCT signal based on the first k-clock signal, and the second sampling module 56 is adapted to sample the respective OCT signal based on the second k-clock signal. The sampling switches between the different k-clocking frequencies. This technique increases the sampling rate and extends the imaging depth.

Referring now to a third embodiment, the controller C may be adapted to acquire or sample the respective OCT signal (indicated by line L1 in FIG. 1) from the OCT device 12 using the internal clock of the digitizer 44, with no k-clock signal, followed by resampling the OCT signal to equally k-spaced samples. The digitizer 44 has an internal clock to indicate when to sample the respective OCT signal. Here, the controller C may employ post-processing interpolation algorithms to resample the data into equally spaced k-space points. This process may involve characterizing the laser sweep function beforehand and using this calibration data to perform the interpolation. For resampling the OCT signal, an example calibration method is to use a mirror (not shown) as the sample and acquire the OCT signal of the mirror with a constant ADC sampling rate. The signal is further processed to obtain wavenumber domain information through Hilbert transform. The obtained wavenumber domain information is used for re-sampling/interpolating the OCT signal to equally wavenumber-spaced samples. The mirror signal may be acquired in advance.

Advancing to block 106 of FIG. 2, the controller C is adapted to generate an output signal indicative of at least one of pupil centering information and tilt angle of the target site based on the first dataset and the second dataset. The tilt angle may be along a depth axis (Z axis), lateral axis (Y axis) or a longitudinal axis (X axis). Advancing to block 108 of FIG. 2, the method 100 includes using the output signal for pre-surgical alignment of the target site 14, in achieving accurate centering and angular adjustment of the delivery optics relative to the entrance pupil of the eye E. As shown in FIG. 2, block 108 may loop back to block 102 in an iterative process. Additionally, the controller C may be adapted to detect shifts in position of the docking device 200 and/or movement of the eye E during surgery. In some embodiments, the docking device 200 is attached directly to the laser delivery optics preoperatively, with both components approaching the eye E simultaneously. In this scenario, concurrent monitoring of both the docking device 200 and the cornea of the patient is tracked by the controller C, as well as the relative distance between them.

Referring to FIG. 3, data from the controller C may be processed through an integrated processor in the laser delivery system 212 and used to optimize a treatment beam 218. The system 10 provides a flexible range OCT device 12 that facilitates tracking and guidance from a considerable distance, reducing dependency on mechanical adjustments of the reference arm 24. In other embodiments, a two-phase docking approach is implemented where the docking device 200 is attached to the patient’s eye, followed by docking the laser delivery system 212 to the docking device 200. Throughout this process, the controller C (via data from the OCT device 12) may be adapted to track specific features within the docking device 200, guiding the movement and angular adjustments of the laser delivery optics as it approaches the docking device 200.

In summary, the system 10 flexibly extends imaging depth for an OCT device 12 and eliminates the need for repeated re-calibrations during post-processing. Digital representation of the analog signals in a swept source OCT device 12 require precise sampling synchronized with the wavelength variations of the swept source laser 16. Conventional approaches for long range OCT imaging operate under the assumption of consistent laser sweep characteristics. However, fluctuations in sweep rate may introduce error in the OCT signal. The system 10 alleviates this issue. By implementing external linear k-space digitizer clocking, the OCT signal acquisition may be obtained without sampling artifacts.

The various components of the system 10 may communicate via a network 70. Referring to FIG. 1, the network 70 may be a Wireless Local Area Network (LAN) which links multiple devices using a wireless distribution method, a Wireless Metropolitan Area Networks (MAN) which connects several wireless LANs or a Wireless Wide Area Network (WAN) which covers large areas such as neighboring towns and cities. Other types of connections may be employed. The network 70 may be a bus implemented in many ways, such as for example, a serial communication bus in the form of a local area network. The local area network may include, but is not limited to, a Controller Area Network (CAN), a Controller Area Network with Flexible Data Rate (CAN-FD), Ethernet, Bluetooth, WIFI and other forms of data connection.

The controller C of FIG. 1 includes a computer-readable medium (also referred to as a processor-readable medium), including a non-transitory (e.g., tangible) medium that participates in providing data (e.g., instructions) that may be read by a computer (e.g., by a computer processor). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic medium, a CD-ROM, DVD, other optical medium, other physical medium with patterns of holes, a RAM, a PROM, an EPROM, a FLASH-EEPROM, other memory chip or cartridge, or other medium from which a computer can read.

Look-up tables, databases, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store may be included within a computing device employing a computer operating system such as one of those mentioned above, and may be accessed via a network in one or more of a variety of manners. A file system may be accessible from a computer operating system and may include files stored in various formats. An RDBMS may employ the Structured Query Language (SQL) in addition to a language for creating, storing, editing, and executing stored procedures, such as the PL/SQL language mentioned above.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A system for guiding an ophthalmic procedure, the system comprising:
an optical coherence tomography (OCT) device configured to obtain respective data of a target site in an eye, the OCT device having a swept source laser tunable through a range of wavelengths, the respective data including a respective OCT signal;
a controller in communication with the OCT device, the controller having a processor and tangible, non-transitory memory on which instructions are recorded, the ophthalmic procedure employing a laser delivery system in communication with the controller;
wherein the OCT device includes a first imaging mode with a first imaging depth and a second imaging mode with a second imaging depth, the first imaging depth being greater than the second imaging depth;
wherein the controller is adapted to:
execute a first sampling module to process the respective OCT signal from the first imaging mode into a first dataset at a first sampling frequency;
execute a second sampling module to process the respective OCT signal from the second imaging mode into a second dataset at a second sampling frequency;
generate an output signal indicative of at least one of pupil centering information and tilt angle of the target site based on the first dataset and the second dataset; and
employ the output signal for pre-surgical alignment of the target site.

2. The system of claim 1, wherein the first imaging depth is between about 30 and 60 millimeters, and the second imaging depth is between about 8 and 30 millimeters.

3. The system of claim 1, wherein:
the first imaging mode and the second imaging mode are executed with a first pulse repetition rate and a second pulse repetition rate, respectively, of the swept source laser;
the controller is adapted to set the first pulse repetition rate to be about half of the second pulse repetition rate when the first sampling frequency and the second sampling frequency are close in value; and
the controller is adapted to set the first pulse repetition rate to be relatively close in value to the second pulse repetition rate when the first sampling frequency is at least twice of the second sampling frequency.

4. The system of claim 3, wherein the first pulse repetition rate is about 50 kilo Hertz and the second pulse repetition rate is about 100 kilo Hertz, the second imaging mode having an equal or higher axial resolution relative to the first imaging mode.

5. The system of claim 1, further comprising:
a docking device adapted to interface between the eye and the laser delivery system;
wherein the first imaging mode is employed when the docking device is in a pre-docking position at a predefined distance from the eye and the second imaging mode is employed when the docking device is in a docking position adjacent to the eye; and
wherein the controller is adapted to align the docking device relative to the target site based in part on the output signal.

6. The system of claim 5, wherein the laser delivery system includes a femtosecond laser device adapted to generate a treatment beam, the controller being adapted to guide the treatment beam towards the target site based on the output signal.

7. The system of claim 1, further comprising:
an interferometer embedded in the OCT device and adapted to transmit a k-clock signal to the controller;
a first analog-to-digital converter and a second analog-to-digital converter operatively connected to the OCT device, the first analog-to-digital converter and the second analog-to-digital converter operating in an interleaved fashion with a predefined delay relative to one another; and
wherein the first analog-to-digital converter is adapted to sample the respective OCT signal based on respective rising edges of the k-clock signal, and the second analog-to-digital converter is adapted to sample the respective OCT signal based on respective falling edges of the k-clock signal.

8. The system of claim 1, wherein:
an interferometer is embedded in the OCT device and adapted to transmit a first k-clock signal and a second k-clock signal to the controller;
wherein the first sampling module is adapted to sample the respective OCT signal based on the first k-clock signal, and the second sampling module is adapted to sample the respective OCT signal based on the second k-clock signal; and
wherein the first k-clock signal is defined by a first frequency and the second k-clock signal is defined by a second frequency different from the first frequency.

9. The system of claim 1, further comprising:
a digitizer operatively connected to the OCT device, the digitizer having an internal clock; and
wherein the controller is adapted to acquire the respective OCT signal from the OCT device using the internal clock of the digitizer, and resample the respective OCT signal to equally k-spaced samples.

10. A method for guiding an ophthalmic procedure in a system having an optical coherence tomography (OCT) device and a controller with at least one processor and at least one non-transitory, tangible memory, the method comprising:
receiving a respective OCT signal from a target site in an eye via a first imaging mode of the OCT device, via the controller, the first imaging mode having a first imaging depth, the OCT device having a swept source laser tunable through a range of wavelengths;
receiving the respective OCT signal from the target site via a second imaging mode of the OCT device, via the controller, the second imaging mode having a second imaging depth, the first imaging depth being greater than the second imaging depth, the ophthalmic procedure employing a laser delivery system in communication with the controller;
executing a first sampling module to process the respective OCT signal from the first imaging mode into a first dataset at a first sampling frequency;
executing a second sampling module to process the respective OCT signal from the second imaging mode into a second dataset at a second sampling frequency;
generating an output signal indicative of at least one of a pupil centering information and tilt angle of the target site based on the first dataset and the second dataset, via the controller; and
aligning a docking device with the target site prior to surgery based on the output signal.

11. The method of claim 10, further comprising:
setting the first imaging depth to be between about 30 and 60 millimeters, and the second imaging depth to be between about 8 and 30 millimeters.

12. The method of claim 10, further comprising:
executing the first imaging mode and the second imaging mode with a first pulse repetition rate and a second pulse repetition rate, respectively, of the swept source laser;
setting the first pulse repetition rate to be about half of the second pulse repetition rate when the first sampling frequency and the second sampling frequency are relatively close in value; and
setting the first pulse repetition rate to be close in value to the second pulse repetition rate when the first sampling frequency is at least twice of the second sampling frequency.

13. The method of claim 12, further comprising:
setting the first pulse repetition rate to be about 50 kilo Hertz and the second pulse repetition rate to be about 100 kilo Hertz, the second imaging mode having an equal or higher axial resolution relative to the first imaging mode.

14. The method of claim 10, further comprising:
positioning a docking device between the eye and the laser delivery system;
employing the first imaging mode when the docking device is in a pre-docking position at a predefined distance from the eye;
employing the second imaging mode when the docking device is in a docking position adjacent to the eye; and
aligning the docking device relative to the target site based in part on the output signal.

15. The method of claim 14, further comprising:
incorporating a femtosecond laser device in the laser delivery system, the femtosecond laser device being adapted to generate a treatment beam; and
guiding the treatment beam to the target site based in part on the output signal.

16. The method of claim 10, further comprising:
receiving a k-clock signal from an interferometer embedded in the OCT device;
coupling a first analog-to-digital converter and a second analog-to-digital converter to the OCT device, the first analog-to-digital converter and the second analog-to-digital converter operating in an interleaved fashion with a predefined delay relative to one another; and
sampling respective rising edges of the k-clock signal via the first analog-to-digital converter; and
sampling respective falling edges of the k-clock signal via the second analog-to-digital converter, via the second sampling module.

17. The method of claim 10, further comprising:
receiving a first k-clock signal from an interferometer embedded in the OCT device, the first k-clock signal being defined by a first frequency;
receiving a second k-clock signal from the interferometer, the second k-clock signal being defined by a second frequency different from the first frequency; and
sampling the first k-clock signal and the second k-clock signal in an interleaved fashion, via the first sampling module.

18. The method of claim 10, further comprising:
coupling a digitizer to the OCT device, the digitizer having an internal clock; and
acquiring an OCT signal from the OCT device using the internal clock of the digitizer, and resampling the OCT signal to equally k-spaced samples.

19. A system for guiding an ophthalmic procedure, the system comprising:
an optical coherence tomography (OCT) device configured to obtain respective data of a target site in an eye, the OCT device having a swept source laser tunable through a range of wavelengths, the respective data including a respective OCT signal;
a controller in communication with the OCT device, the controller having a processor and tangible, non-transitory memory on which instructions are recorded, the ophthalmic procedure employing a laser delivery system in communication with the controller;
a docking device configured to interface between the eye and the laser delivery system;
wherein the OCT device includes a first imaging mode with a first imaging depth and a second imaging mode with a second imaging depth, the first imaging depth being greater than the second imaging depth;
wherein the first imaging mode and the second imaging mode are executed with a first pulse repetition rate and a second pulse repetition rate, respectively, of the swept source laser;
wherein the first imaging mode is employed when the docking device is in a pre-docking position at a predefined distance from the eye and the second imaging mode is employed when the docking device is in a docking position adjacent to the eye;
wherein the controller is adapted to:
execute a first sampling module to process the respective OCT signal from the first imaging mode into a first dataset at a first sampling frequency;
execute a second sampling module to process the respective OCT signal from the second imaging mode into a second dataset at a second sampling frequency;
generate an output signal indicative of at least one of a pupil centering information and a tilt angle of the target site based in part on the first dataset and the second dataset; and
employ the output signal for pre-surgical alignment of the docking device relative to the target site; and
wherein the controller is adapted to set the first pulse repetition rate to be about half of the second pulse repetition rate when the first sampling frequency and the second sampling frequency are relatively close in value.

20. The system of claim 19, wherein the first imaging depth is between about 30 and 60 millimeters, and the second imaging depth is between about 8 and 30 millimeters, the controller being adapted to set the first pulse repetition rate to be relatively close in value to the second pulse repetition rate when the first sampling frequency is at least twice of the second sampling frequency.
